(19) United States
(12) Patent Application Publication
(10) Pub. No.: US 2025/0036271 A1
(43) Pub. Date: Jan. 30, 2025

(54) SYSTEMS AND METHODS FOR EFFICIENTLY ZOOMING AND SCROLLING ALERTS TIMELINE

(71) Applicant: KONINKLIJKE PHILIPS N.V., EINDHOVEN (NL)

(72) Inventors: Johannes Henricus Maria Korst, EINDHOVEN (NL); Serverius Petrus Paulus Pronk, VUGHT (NL); Rithesh Sreenivasan, BENGALURU (IN); Jurgen Jan Rusch, EINDHOVEN (NL); Mauro Barbieri, EINDHOVEN (NL)

(21) Appl. No.: 18/776,317

(22) Filed: Jul. 18, 2024

Related U.S. Application Data
(60) Provisional application No. 63/528,667, filed on Jul. 25, 2023.

(51) Int. Cl. G06F 3/04845; G06F 3/04847; G16H 40/20

(52) U.S. Cl. CPC G06F 3/04845; G06F 3/04847; G16H 40/20; G06F 2203/04806

(57) ABSTRACT

An alert monitoring method includes receiving alerts generated by one or more medical systems, each alert being timestamped and associated to the medical system that generated the alert; segmenting a displayed time span into discrete time units whose size depends on the displayed time span; generating a timeline representing the alerts over the displayed time span, the timeline comprising a two-dimensional grid of blocks with each block having a corresponding discrete time unit and a corresponding alert source for the one or more medical systems, and each block being color coded based on a fraction of the corresponding discrete time unit over which the corresponding alert is critical; displaying, on a display device, the timeline representing the alerts over the displayed time span; and in response to receiving a user input adjusting the displayed time span, displaying an updated timeline representing the alerts over the updated displayed time span.

### CROSS REFERENCE TO RELATED APPLICATIONS

This application claims the benefit of U.S. Provisional Patent Application No. 63/528,667 filed Jul. 25, 2023. This application is hereby incorporated by reference herein.

### FIELD

The following relates generally to the device and systems monitoring arts, the radiology arts, electronic database maintenance arts, radiology database maintenance arts, picture archiving and communication system (PACS) maintenance arts, and related arts.

### BACKGROUND

The medical images that are produced by medical imaging systems, such as magnetic resonance (MR), computed tomography (CT), and image guided therapy (IGT) systems, are stored for later viewing in a secure storage system commonly called a Picture Archiving and Communication System (PACS). Radiologists access and interpret the images from a PACS to report their findings in a so-called imaging or radiology report. If images are not accessible because a PACS is down, or if loading images from a PACS is slow due to a degraded performance, then radiologists are not able to do their work efficiently. Also, the work of other users of a PACS, such as biomedical engineers (“bio-meds”), technicians and referring physicians, will be adversely affected by a degraded performance of a PACS. Consequently, it should be avoided that a PACS experiences a reduced functionality, whenever possible. Nowadays, a PACS is built using standard computer technology, where often virtual machine technology is used to achieve hardware platform independency and to allow easy redirecting of virtual processing nodes when the underlying hardware platform experiences problems.

To proactively avoid unplanned down time of a medical database, such as a PACS, a monitoring system can repeatedly check specific parameter values (such as central processing unit (CPU), memory and disk usage as well as queue lengths) for the various nodes present in a PACS. The monitoring system can generate an alert whenever a parameter value exceeds a WARNING or CRITICAL threshold. These alerts are sent to a central location and presented on a dashboard that is used by a proactive monitoring team. Using dedicated business rules, the proactive monitoring team members can use these alerts to create tickets/cases that are next handled by remote service engineers.

The alerts can be characterized by the site and device at which they are generated, as well as by an alert type specifying the type of parameter (CPU, memory, disk, etc.). In addition, an alert will typically contain additional information on the specific hardware parameter that is monitored as well as the parameter values that are observed. A site may be a small hospital or a large hospital or even a large hospital chain consisting of multiple locations.

The successive alerts for a given combination of site, host and alert type can be represented as a concatenation of “ok,” “warning,” and “critical” intervals. A critical interval is a maximum-length sequence of critical alerts that are not interleaved with warning or ok alerts. It starts with a first critical interval, and it ends with the first non-critical alert that succeeds the last critical alert in the given sequence. Warning and ok intervals are defined similarly. By coloring, for example, a critical interval red, a warning interval orange (or yellow), and an ok interval green, a heat-map type of a time line can be obtained. To identify individual issues or long-term trends, it may be necessary to inspect all critical intervals presented in the time line over a longer period of time.

Critical intervals may have a short duration, possibly lasting only a few seconds. In order to be able to see each individual critical interval, one has to zoom in on a time line to a sufficient level of detail such that each critical interval is clearly visible. For example, one can zoom in to such a level that each column of pixels of the display on which the time line is inspected corresponds to, for example, a second of the time line. On a full high-definition (HD) screen, the number of pixels in a horizontal direction is 1,920. If one pixel represents one second, then 1,920 pixels represents 32 minutes. Hence, inspecting a time line of one week of data at this zoom level would result in showing only 32 minutes at a time of the total of 10,080 minutes contained in a single week. Given that 10,080/32=315, only a fraction of 1/315 (≈0.32%) of the complete time line at a time could be seen at this zoom level. Hence, an excessive amount of scrolling would be needed to make sure that no critical alert is missed. Furthermore, at this detailed level there would not be a good overview of the alerts, such as seeing trends of slowly increasing parameter values.

In addition, it often happens that critical intervals started almost simultaneously for multiple parameters. It can be beneficial to know for which parameter the critical interval started first. This can help find the root cause of the issue at hand. The time lines may be relatively far away from each other in vertical distance. One would have to zoom in on the timeline considerably to clearly see which one starts first. And even then, supporting vertical lines should be drawn sometimes to precisely see which one starts first. However, drawing these supporting vertical lines can clutter the image.

The following discloses certain improvements to overcome these problems and others.

### SUMMARY

In some embodiments disclosed herein, a non-transitory computer readable medium stores instructions readable and executable by an electronic processor to perform an alert monitoring method. The alert monitoring method includes receiving alerts generated by one or more medical systems, each alert being timestamped and associated to the medical system that generated the alert; segmenting a displayed time span into discrete time units whose size depends on the displayed time span; generating a timeline representing the alerts over the displayed time span, the timeline comprising a two-dimensional grid of blocks with each block having a corresponding discrete time unit and a corresponding alert source for the one or more medical systems, and each block being color coded based on a fraction of the corresponding discrete time unit over which the corresponding alert is critical; displaying, on a display device, the timeline representing the alerts over the displayed time span; and in response to receiving a user input adjusting the displayed time span, repeating the segmenting, generating, and displaying to display an updated timeline representing the alerts over the updated displayed time span.

In some embodiments disclosed herein, an alert monitoring method includes receiving timestamped alerts generated by a plurality of alert sources; segmenting a time span to be displayed into discrete time units whose size depends on the time span; displaying, on a display, a timeline representing the alerts over the time span, the displayed timeline comprising blocks having corresponding discrete time units and alert sources, each block being displayed with a color determined based on a fraction of the corresponding discrete time interval over which the corresponding alert is critical; receiving an adjusted time span to be displayed; segmenting the adjusted time span into adjusted discrete time intervals whose size depends on the adjusted time span; and displaying an updated timeline representing the alerts over the adjusted time span, each block of the updated timeline being displayed with a color determined based on a fraction of the corresponding adjusted discrete time interval over which the corresponding critical alert.

In some embodiments disclosed herein, a non-transitory computer readable medium stores instructions readable and executable by an electronic processor to perform an alert monitoring method. The alert monitoring method includes segmenting a displayed time span into discrete time units whose size depends on the displayed time span; generating a timeline representing alerts generated by alert sources over the displayed time span, each alert being timestamped and associated to the alert source that generated the alert, the timeline comprising a two-dimensional grid of blocks with each block having a corresponding discrete time unit and a corresponding alert source comprising one of the alert sources or a group of two or more of the alert sources, and each block being color coded based on a fraction of the corresponding discrete time unit over which the corresponding alert is critical; displaying, on a display device, the timeline representing the alerts over the displayed time span; and in response to receiving a user input adjusting the displayed time span, repeating the segmenting, generating, and displaying to display an updated timeline representing the alerts over the updated displayed time span.

One advantage resides in visualizing a number of alerts per time unit for an extended time period in a way that facilitates both visualization of individual alerts and groups or trends in the alerts.

Another advantage resides in providing a timeline decluttered from lines between time units of the timeline.

Another advantage resides in showing trends between different levels of alerts.

Another advantage resides in providing color-coded alerts on a timeline based on a severity level of the alerts.

A given embodiment may provide none, one, two, more, or all of the foregoing advantages, and/or may provide other advantages as will become apparent to one of ordinary skill in the art upon reading and understanding the present disclosure.

### BRIEF DESCRIPTION OF THE DRAWINGS

The disclosure may take form in various components and arrangements of components, and in various steps and arrangements of steps. The drawings are only for purposes of illustrating the preferred embodiments and are not to be construed as limiting the disclosure.

FIG. 1 diagrammatically illustrates an alert monitoring apparatus in accordance with the present disclosure.

FIG. 2 diagrammatically illustrates an alert monitoring method using the apparatus of FIG. 1.

FIG. 3-7 show examples of timelines generated by the apparatus of FIG. 1.

### DETAILED DESCRIPTION

Embodiments disclosed herein relate to monitoring of alerts from a Picture Archiving and Communication System (PACS) or other complex medical system or set of systems with many electronically generated and/or monitored parameters that can be subject to approaching entering critical states resulting in automated issuance of corresponding warning alerts and critical alerts. For example, a given parameter may be in a critical alert state if the value exceeds some parameter-specific upper and/or lower threshold, and may be in the warning alert state if its value is sufficiently close to the upper or lower threshold. In such systems, a timeline of alerts is maintained, usually with time on the abscissa and rows corresponding to the monitored parameters. Using such a timeline visualization, alerts can be shown as vertical lines, and hence maintenance personnel can easily observe clusters of alerts and visually recognize patterns in timing and parameters involved in system malfunctions. To assist in this, the timeframe shown on the display can be zoomed in or out, and when zoomed in the window of the visualization can be scrolled horizontally or vertically.

However, there are some problems with this approach. The number of alerts can be very large, numbering in the hundreds, thousands, or more. When zoomed out, it can become difficult or impossible to depict and visually discern individual alerts. Zooming in can enable focusing on specific alerts, but at the expense of losing the larger picture provided by the zoomed-out visualization. This can be particularly problematic since it may be that one or a few isolated alerts some time back started a cascade of alerts, so that locating those early isolated alerts illuminates the root cause. However, in the zoomed out view the few early isolated alerts may not be visible, while in the zoomed in view of the cascade of alerts the few early isolated alerts are outside of the zoomed-in window. As another example, an infrequent but gradually increasing rate of alerts may not be identifiable in the zoomed out view due to their infrequency, but the slow trend will also not be discernable in the zoomed in view if the zoomed window is too small to capture the slow trend.

Embodiments disclosed herein provide visualization to more easily see which alert is first, assisted by the time unit boundaries (which may be shown only whenever relevant, since in some embodiments only when there are differences between successive time units the boundary will be shown as a boundary between different colors). Additionally, if two critical intervals start at the same time unit and continue for several time units, then the colors of the first time units at which the critical intervals start will often show which critical interval started first. The one for which the first time unit will have a color closest to red, will have started earlier.

Moreover, if a parameter frequently changes between an ok, warning, or critical state (or more generally, between various alert levels), then it is difficult to see a trend in such a seemingly chaotic pattern. It can be important to make a distinction between almost random patterns and patterns for which the time that the given parameter is in a critical state is slowly growing over time. In that case, showing the critical fraction of successive relatively large time units, of say 15 minutes, 1 hour, or even 6 hours, could more clearly show a transition towards more red colors for the successive time units. To address such problems, the following discloses an approach in which the timelines for the respective parameters are segmented into discrete time units. Each time unit of a given parameter timeline is color-coded based on the fraction of the total time of that time unit that the corresponding parameter is in the critical alert state. That is, for each time unit the fraction of time in the critical alert state is mapped to a color, and that time unit of the timeline is color coded by that mapped color. For example, a small fraction of time being in the critical alert state may map to green, while a large fraction of time being in the critical alert state may map to red. The mapping may be continuous from fraction=0 to fraction=1, or may be discretized. Since it can be important to distinguish no alerts from very few alerts, a special color mapping (e.g., a special shade of green) may be used to indicate the parameter is never in the critical alert state over the time unit, and another special color (e.g., a special shade of red) may be used to indicate the parameter is in the critical alert state for fully 100% of the time unit.

The color coding is dynamically adjusted based on the zoom level. This is done by automatically changing the time unit into which the parameter timelines are segmented based on the zoom level. Specifically, in response to zooming in, the time unit is reduced (unless it is at an absolute minimum value), while in response to zooming out, the time unit is increased. Optionally, the color mapping may also be adjusted based on the level of zoom. By this mechanism, the color coding effectively highlights relatively infrequent alerts when zoomed out so that they are visually detectable, and automatically adjusts to only highlight more frequent alerts when zoomed in. In another example, the relatively infrequent alerts can be identified by zooming in on the timeline and adjusting a time unit of the timeline.

In another variant, the vertical rows of the visualization corresponding to parameters can be hierarchically folded in or out. This enables the user to obtain an overview and then drill down to identify the problem. In another variant, the user can rearrange the vertical rows manually using drag-and-drop graphical user interface (GUI) operations or the like.

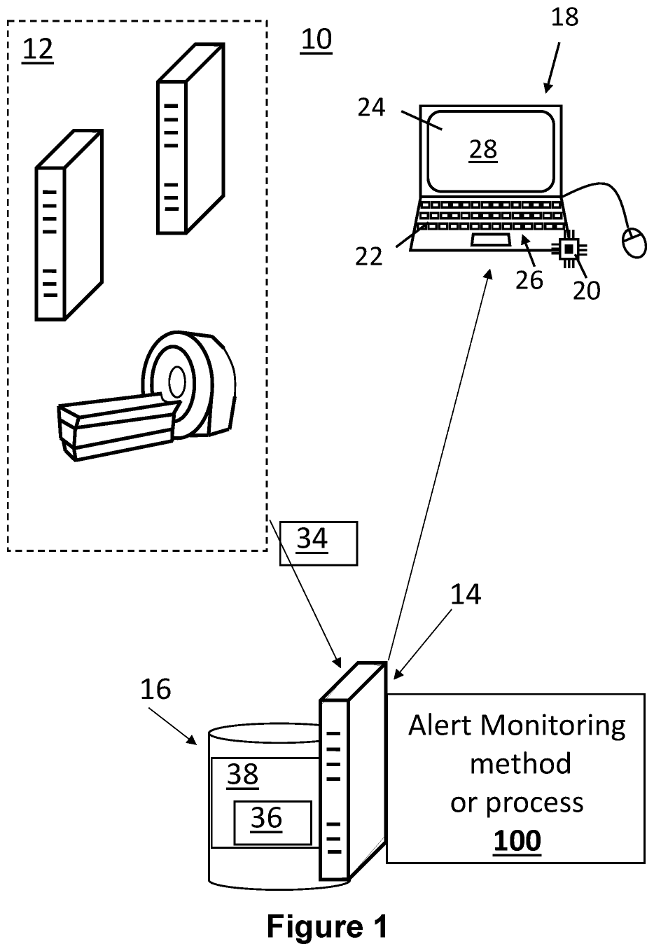

With reference to FIG. 1, an illustrative apparatus 10 for monitoring alerts from one or more associated medical systems 12 is shown. The medical systems 12 can comprise, for example, n medical databases 12 servicing respective medical entities. While two such databases 12 are shown in FIG. 1, any suitable number of n medical databases 12 can be included, and in some embodiments n may be on the order of dozens, hundreds, or more medical systems. In some embodiments, the n medical databases 12 comprise n respective picture archiving and communication system (PACS) databases, although this is merely an example and the medical databases 12 may in general be assigned other nomenclatures. For example, a medical database servicing a medical entity which is a cardiology department might be a cardiovascular information system (CVIS). As another example, a medical database servicing a medical entity might be an integrated PACS/RIS system where “RIS” refers to “Radiology Information System.” These are merely further examples. Each PACS or other medical database 12 typically comprises a server computer running suitable software implementing the PACS, either running directly on the server (i.e., running on silicon) or running in a virtual machine (VM) that runs on the server. By way of nonlimiting illustrative example, a medical database 12 could comprise a server computer running the IntelliSpace PACS (available from Koninklijke Philips N.V., Eindhoven, the Netherlands). In another example, the medical systems 12 can comprise, for example, n medical imaging devices 12 and/or components thereof. While one such imaging device 12 are shown in FIG. 1, any suitable number of n medical imaging devices 12 can be included, and in some embodiments n may be on the order of dozens, hundreds, or more medical imaging devices. The medical device(s) 12 can include, for example, magnetic resonance (MR), computed tomography (CT), image guided therapy (IGT) systems, ultrasound, X-ray, positron emission tomography (PET), single photon emission computed tomography (SPECT), and so forth.

As shown in FIG. 1, the monitoring apparatus 10 includes, or is accessible by, a server computer 14 typically disposed remotely from the medical systems 12. The server computer 14 comprises a computer or other programmable electronic device that includes or has operative access to a non-transitory computer readable medium. The server computer 14 is generally (although not necessarily) separate from any of the server computers implementing the n medical systems 12, and the server computer 14 typically communicates with the n medical systems 12 via the Internet, optionally along with one or more local area networks (LAN) or wide-area networks (WAN), such as a LAN or WAN of the hospital at which the medical systems 12 is located, and/or a LAN or WAN of the vendor or of a cloud computing service provider providing the server computer 14 to the vendor. It will be appreciated that the server computer 14 could be implemented as a plurality of server computers, e.g., interconnected to form a server cluster, cloud computing resource, or so forth, to perform more complex computational tasks.

An electronic processing device 18, such as a workstation computer, or more generally a computer, a mobile device (e.g., a tablet computer), is operable by a service engineer (SE), information technology (IT) professional, or the like to provide a user interface with an alert monitoring method or process 100 running on the server computer 14. The electronic processing device 18 includes typical components for a user interfacing computer, such as an electronic processor 20 (e.g., a microprocessor), at least one user input device (e.g., a mouse, a keyboard, a trackball, and/or the like) 22, and a display device 24 (e.g., an LCD display, plasma display, cathode ray tube display, and/or so forth). In some embodiments, the display device 24 can be a separate component from the electronic processing device 18, or may include two or more display devices. To display color-coded image(s), the display device 24 should preferably be a color display device.

The non-transitory storage media 16 may, by way of non-limiting illustrative example, include one or more of a magnetic disk, RAID, or other magnetic storage medium; a solid-state drive, flash drive, electronically erasable read-only memory (EEROM) or other electronic memory; an optical disk or other optical storage; various combinations thereof; or so forth; and may be for example a network storage, an internal hard drive of the electronic processing device 18, various combinations thereof, or so forth. It is to be understood that any reference to a non-transitory medium or media 16 herein is to be broadly construed as encompassing a single medium or multiple media of the same or different types. Likewise, the electronic processor 20 may be embodied as a single electronic processor or as two or more electronic processors. The non-transitory storage media 16 stores instructions executable by the at least one electronic processor 20. The instructions include instructions to generate a visualization of a graphical user interface (GUI) 28 for display on the display device 24.

The medical systems 12 communicate with the server computer 14 via a communication link, which typically comprises an electronic network including the Internet augmented by local area networks (e.g. LAN or WAN) for electronic data communications. The electronic processing device 18 may be a dumb terminal connected with the server computer 14 via a LAN, WAN, Internet, or so forth. In the illustrative example, the server computer 14 handles the communication with the n medical systems 12 and the computation of the heat map representing alert counts as disclosed herein, and the electronic processing device 18 primarily serves as the user interfacing device including displaying a color-coded timeline on the display 24. However, this is merely an illustrative example, and the processing may be variously distributed between the server 14 and electronic processing device 18. In some embodiments, only one computer may be provided which includes all functionality—e.g., the server computer 14 could be omitted and its functionality performed solely by the electronic processing device 18.

The apparatus 10 is configured as described above to perform an alert monitoring method or process 100 of monitoring alerts 34 received from the medical system(s) 12. The database 16 stores instructions executable by the server computer 14 (and/or by the electronic processing device 18) to perform the alert monitoring method or process 100 which includes presenting a timeline 38 representing the alerts 34 over a time span. In some examples, the method 100 may be performed at least in part by cloud processing (that is, the server computer 16 may be implemented as a cloud computing resource comprising an ad hoc network of server computers).

To proactively avoid unplanned slowdowns, malfunctions, down time, or other issues with or of the medical systems 12, the monitoring apparatus 10 can repeatedly check specific parameter values (such as CPU, memory disk usage, queue lengths, and so forth) for the various nodes (i.e., medical devices) present in the medical systems 12. The monitoring apparatus 10 can generate an alert 34 if a value of a performance parameter for the medical system 12 meets a specified criterion, such as whenever a parameter value exceeds a predetermined threshold. These alerts 34 are sent to the server computer 14 and/or the electronic processing device 18, and presented on the GUI 28. A user can use these alerts 34 to create tickets/cases that are next handled by remote service engineers.

The alerts 34 can be characterized by the location and device at which they are generated, as well as by an alert type specifying the type of parameter (CPU, memory, disk, etc.). In addition, the alert 34 can typically contain additional information on the specific hardware parameter that is monitored as well as the parameter values that are observed. A site may be a small hospital or a large hospital or even a large hospital chain consisting of multiple locations. An alert may be present or active for a certain duration. For example, in the case of a monitored PACS, if total CPU usage of the server or virtual machine (VM) hosting the PACS exceeds some designated percentage (e.g., 80% usage) then a CPU overload alert may be triggered, and this CPU overload alert will remain active until the CPU usage drops back below the threshold (e.g., drops back below 80%). If the alerts 34 are monitored at fixed sampling intervals, e.g., every second, then these samples will indicate the CPU overload alert for each time sample where the CPU usage exceeds the overload threshold. This is merely a non-limiting illustrative example.

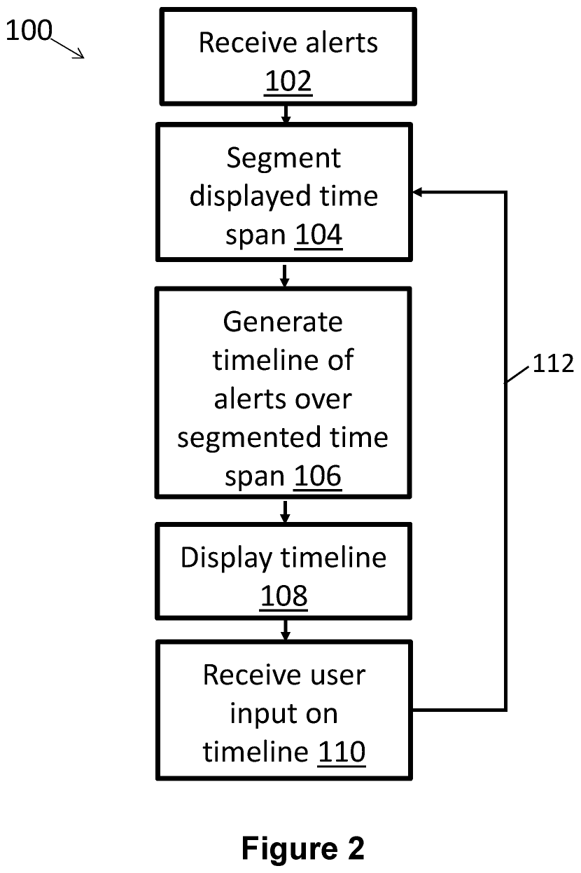

With reference to FIG. 2, and with continuing reference to FIG. 1, an illustrative embodiment of an instance of the alert monitoring method 100 is diagrammatically shown as a flowchart.

At an operation 102, alerts 34 generated by the medical systems 12 are received at the server computer 14 via an electronic network (i.e., the Internet). Each alert 34 is timestamped with a receipt time when the alert 34 was received at the server computer 14, and each alert 34 is associated to the medical system 12 that generated the alert 34.

At an operation 104, a displayed time span T displayed on the display device 24 is segmented into discrete time units whose size depends on the displayed time span. For example, the displayed time span T can be segmented into seconds, minutes, days, weeks, months, years, and so forth.

At an operation 106, a timeline 38 represents the alerts 34 over the time span T with a grid of blocks 36. Each block 36 corresponds to a time interval with the length of one time unit and has a corresponding alert source comprising one of the medical systems or a group of two or more of the medical systems 12. Each block 36 is color coded based on the fraction of time during this time interval that the alert 34 is critical. To construct the timeline 38, each block 36 of the timeline 38 is color coded based on the fraction of time during this time interval that the alert is critical. As used herein, the term “color” (and variants thereof) can refer to different discrete colors (e.g., red, green, blue, etc.), different shades (e.g., light blue or dark blue), different hues, different brightness levels (e.g., “bright” red vs. “dim” red), or so forth. For example, the fraction-to-color mapping is a continuous fraction-to-color mapping, and/or the fraction-to-color mapping depends on the displayed time span.

In some embodiments, the color scale includes a first color (i.e., red), a second color (i.e., yellow), and a third color (i.e., green). The mapping operation 106 includes color-coding, with a first color, the blocks 36 whose fraction of the corresponding discrete time interval over which the corresponding alert is critical is in a first range (e.g., blocks 36 having a high number of alerts 34 can be color-coded red). Blocks 36 whose fraction of the corresponding discrete time unit over which the corresponding alert is critical is in a second range can be color-coded with a second color (e.g., blocks 36 having an intermediate number of alerts 34 can be color-coded yellow). Blocks 36 whose fraction of the corresponding discrete time unit over which the corresponding alert is critical is in a third range can be color-coded with a third color (e.g., blocks 36 having a low number of alerts 34 can be color-coded green). In some examples, the color scale further includes a fourth color (e.g., white), and the mapping operation 106 includes color-coding blocks 34 whose fraction of the corresponding discrete time unit over which the corresponding alert is critical is equal to zero with the fourth color. In other examples, the mapping operation 106 includes color coding, with a max-indicative color, blocks 36 whose fraction of the corresponding discrete time unit over which the corresponding alert is critical is equal to unity. This is merely an illustrative example, and the number of colors in the color scale could be different. Moreover, while the illustrative color scale is described in terms of values of hue (e.g., red, green, yellow, magenta, et cetera) it is to be understood that the color scale can include scaling on the basis of various color properties or combinations of properties, including hue, tint, brightness, shade, saturation, or so forth.

At an operation 108, the timeline 38 is displayed on a color display (i.e., on the GUI 28 of the display device 24). In some embodiments, the timeline 38 can be used to predict a potential failure of one or more of the medical systems 12. For example, if a number of alerts 34 generated by one of the medical systems 12 are critical as shown in the timeline 38, then that medical system 12 may be at risk of malfunction or failure. In another example, the timeline 38 can be used to predict a trend (i.e., a commonality) in the colors over time for a particular medical system 12 (e.g., illustrating the max-indicative color for an extensive period of time).

In an operation 110, a user can select to modify the displayed timeline 38 via the at least one user input device 22. This operation 110 can include a variety of examples. In one example, in response to receiving a user input adjusting the displayed time span (e.g., by zooming in or out), process flow passes back as indicated by flowback arrow 112 so that the segmenting, generating, and displaying operations 104, 106, 108 are repeated to display an updated timeline 38 representing the alerts 34 over the updated displayed time span. Such updating can also occur as new alerts arrive via the operation 102. For example, in some embodiments the time span represents a sliding time window.

In one example of the operation 110, a zoom control is provided on the GUI 28 via which a user can zoom the displayed timeline in or out. For example, the zoom control can be implemented by “+” and “−” buttons shown on the display, where user selection (e.g., pressing) of the “+” button zooms in (i.e., reduces the time span) while user selection of the “−” button zooms out (i.e. increases the time span). In another example, if the display 24 is a touch-sensitive display then the GUI may be designed with a pinch zoom, in which the user can: zoom in by pressing two (or possibly more) fingers to the display 24 and spreading them apart; and zoom out by pressing two (or more) fingers to the display 24 and bringing them together. These are merely nonlimiting illustrative examples of some suitable zoom inputs. Zooming typically occurs with a stationary point on the display 24 (i.e., a position of a mouse cursor). The user input adjusting the displayed time span is received via the zoom control. In another example, this input is received at the server computer 14, and a list of the alerts 34 received from the medical system 12 corresponding to a chosen point (e.g., the position of a cursor of a mouse comprising the user input device 22) is displayed on the display device 24. In another example, a color scale of the block 36 selected by the user can be adjusted in response to receiving the user input. To do so, the adjusting can include automatically changing the discrete time unit into which the parameter timelines are segmented based on a determined zoom level. In another example, a user input can be received that modifies the alert sources by grouping two or more of the medical systems 12 into a single alert source or breaking up an alert source comprising two or more of the medical systems into two or more separate alert sources, and the segmenting, generating, and displaying operations 104, 106, 108 are repeated the to display an updated timeline 38 representing the alerts 34 with the modified alert sources. In another example, the adjusting can include determining a color of the blocks 36 when multiple sources of alerts 34 are combined (e.g., averaging fractions of the alerts 34, taking a maximum value of the alerts 34, and so forth). These are merely illustrative examples and should not be construed as limiting.

### Example

The following describes the apparatus 10 and the method 100 in more detail. The apparatus 10 is configured to visualize the number of alerts per time interval per site for the various sites in one overall dashboard, by using a site-dependent transformation.

The system 10 is configured to dynamically adapt the visualization of the alerts timeline 38 to the chosen zoom level so that, irrespective of the zoom level, critical intervals always remain detectable. To realize this, multiple time lines 38 can be constructed at various levels of time units, the critical, warning, and ok intervals are not represented. Instead, the intervals are represented as a color, from a continuous (i.e., green, yellow, red) range, the time that the given alert 34 is critical as a fraction of the given time unit, for the successive time units. The time unit for which the fraction of critical time is computed will be adapted to the chosen zoom level, so that a short critical interval surrounded by longer time intervals are still detectable as it will result in a color for the time unit in which it is contained that differs considerably from the color that is used for the surrounding time units that do not contain a critical time interval.

Some examples of the time units can include, for example, 1 week, 1 day, 6 hours, 1 hour, 15 minutes, 5 minutes, 1 minute, 15 seconds, 5 seconds, and 1 second. Hence, for the timeline 38 with a granularity of one week, the fraction of time that the given alert is critical for each of the successive weeks would be shown. Likewise, for the timeline 38 with a granularity of one day, the fraction of time that the given alert is critical for each of the successive days would be shown, and so forth.

To be able to clearly see the difference between a time interval that contains a small critical interval and a time interval that does not contain any critical intervals, a dark green color can be used for a time unit that does not overlap with critical intervals and use a brighter color green for a time unit that has a small overlap with one or more critical intervals, however small the overlap. In the same manner, a dark red color can be chosen for a time unit for which the alert 34 is continuously critical and a brighter color red can be chosen for a time unit that has a large overlap with the critical intervals.

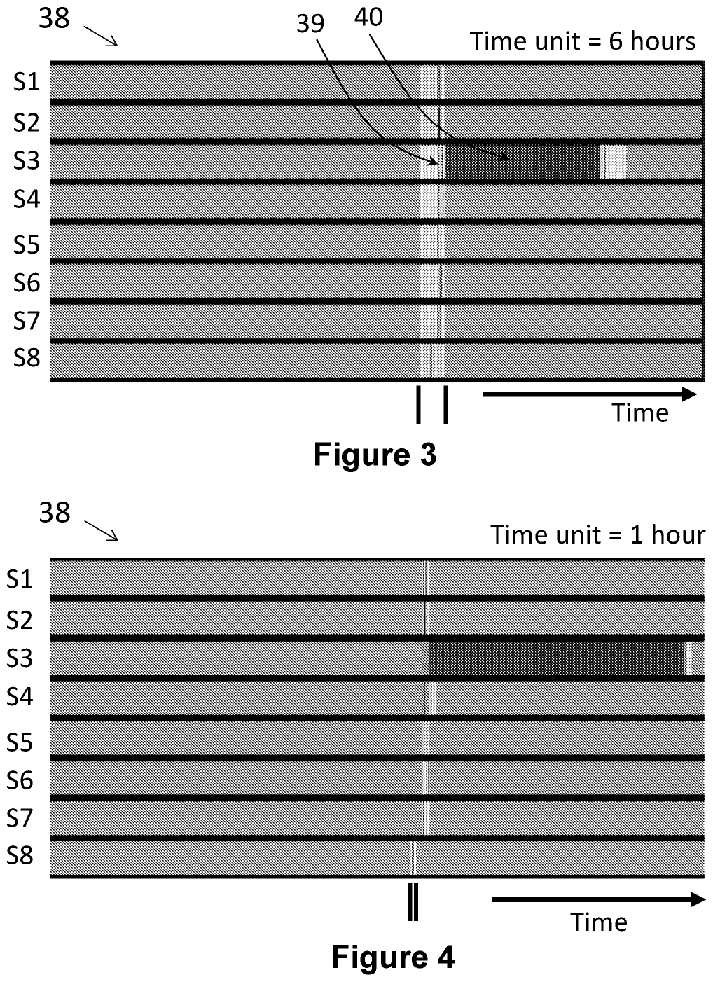

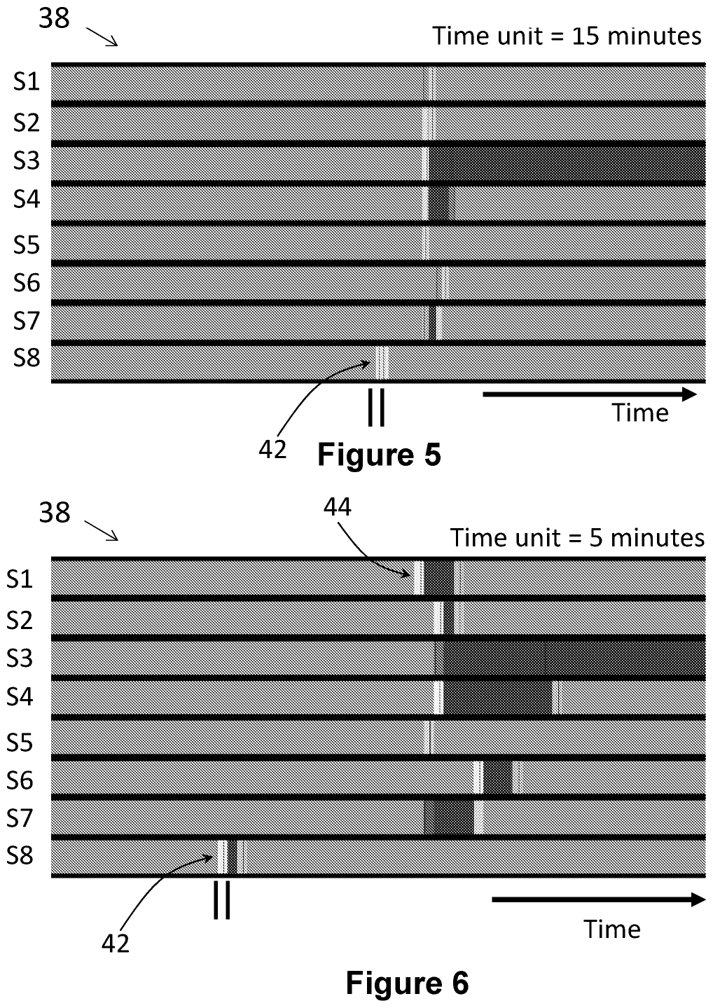

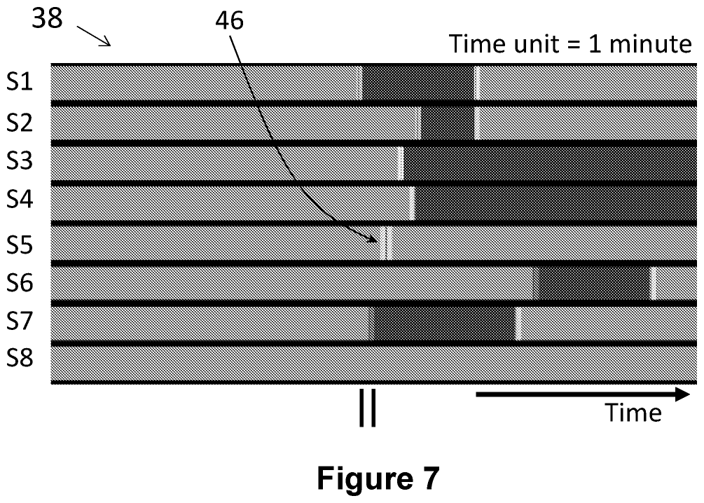

With reference to FIGS. 3-7, a nonlimiting illustrative example is shown of how the displayed timeline 38 changes with successive zoom-in operations, leading to the time unit changing from 6 hours (FIG. 3), to 1 hour (FIG. 4), to 15 minutes (FIG. 5), to 5 minutes (FIG. 6), and finally to 1 minute (FIG. 7). A succession of zoom-out operations can conversely be visualized by going from FIG. 7 to FIG. 3, i.e., zooming out from 1 minute (FIG. 7) to 5 minutes (FIG. 6) to 15 minutes (FIG. 5) to 1 hour (FIG. 4) to 6 hours (FIG. 3). The successive zoom displays for the zoom-in sequence are described in more detail below. In the example timelines of FIGS. 3-7, there are eight alert sources being monitored. In FIGS. 3-7 the alerts of each alert source being monitored is shown on a separate horizontal row of the displayed timeline, and the eight alert sources thus correspond to eight horizontal rows of the timeline which are labeled by labels “S1”, “S2”, “S3”, “S4”, “S5”, “S6”, “S7”, and “S8” in each of FIGS. 3-7. Each alert source “S1”-“S8” could be a medical system (including a subsystem of a larger medical system, such as a component of an MRI scanner), or each alert source “S1”-“S8” could be an alert associated with a particular metric of a system being monitored, e.g. in the case of monitoring a PACS the alert source “S1” could be associated with CPU load, the alert source “S2” could be associated with data storage writing rate, the alert “S3” could be associated with consumed bandwidth, and so forth.

Hence, in this example, alerts from alert source “S1” might be generated when the monitored CPU load exceeds 80%, alerts from alert source “S2” might be generated when the monitored data storage writing rate exceeds some set threshold, and so forth. It will be appreciated that in a given implementation these generic numeric labels “S1”-“S8” might be replaced by semantically informative labels such as “CPU load”, “Write rate”, “Bandwidth”, and so forth. In other examples, the alert sources could be an MRI scanner or components thereof, patient monitors that are monitoring one or more patients, or so forth. Furthermore, the illustrative eight alert sources is a nonlimiting example, and the number of monitored alert sources could be substantially larger, e.g., in the dozens, hundreds, or more. In the timelines 38 of FIGS. 3-7, time is plotted on the abscissa or X-axis, as annotated in each of FIGS. 3-7. It will be appreciated that this “Time” label and corresponding arrow is provided in each of FIGS. 3-7 for clarity of exposition, may be omitted in the displayed timeline 38 in an actual implementation as it will be implicitly understood by the user. In FIGS. 3-7, different colors of the color-coded blocks are diagrammatically indicated by different types of hatching.

FIG. 3 shows a first example of a timeline 38, in which the selected time unit is 6 hours. The timeline 38 of FIG. 3 has a total time span of several days, with the timeline for each monitored medical device (or alert source) “S1”-“S8” divided into time units of 6 hours each. Each time unit of 6 hours for each medical device/alert source “S1”-“S8” is colored uniformly with a color indicative of a fraction of the corresponding discrete time unit (here a 6-hour discrete time unit) over which the corresponding alert is critical. As shown in FIG. 3, there is an issue during a single interval of 6 hours corresponding to the time block for each medical device at the location of the time ticks annotated below the timeline 38 in FIG. 3. This issue is visually recognizable due to the different color coding (diagrammatically indicated by different hatching) for these blocks (labeled by reference number 40 in FIG. 3). For the medical device “S3”, the issue remains critical for several successive time units thereafter, each of 6 hours in duration, as indicated by the different hatching for six successive time units. This different hatching might, for example, correspond to these blocks 40 having a dark red color, meaning that the issue is continuously critical for at least 36 hours. The thin vertical black lines (one of which in row “3” is labeled by reference number 39 in FIG. 3 as an example) specify the moments in time that an alert was generated. Such thin vertical black lines in the same timeline may overlap in time so that, depending on the zoom level, individual alerts 34 may not be individually visible. In such cases, the vertical lines may optionally not be shown, instead relying on the color coding. This is the case for the blocks 40, where the dark red (or other chosen) color indicates a continuously critical state for the device “S3” over this time interval.

FIG. 4 shows the timeline 38 after zooming in. For the resulting reduced time span of the zoomed-in timeline 38 of FIG. 4, the segmentation into time unit is changed by using smaller time units in which the selected time unit is 1 hour (zoomed in from the 6 hour timeline of FIG. 3). As shown in FIG. 4, as indicated by the time between the two time ticks below the timeline 38 of FIG. 4, for the time line of device “S8” (the bottom one), the issue started approximately 2 hours earlier than for the other time lines as indicated by the portion 42 of the timeline for alert source “S8”. This could indicate that the root cause is related to the parameter tracked by the alert source of time line “S8”.

FIG. 5 shows the timeline 38 after further zooming, in which the selected time unit is now 15 minutes (zoomed in from the 1 hour timeline of FIG. 4). As shown in FIG. 5, as indicated by the time between the two time ticks below the timeline 38, a better view of the alerts 42 from alert source “S8” can be shown.

FIG. 6 shows a still further zoomed timeline 38, in which the selected time unit is now 5 minutes (zoomed in from the 15 minute timeline of FIG. 5). As shown in FIG. 6, as indicated by the time between the two time ticks, the time between the issue 42 at the time line for alert source “S8” and the issues at the other time lines is probably too large to make them related. Discarding the issue 42 at time line of source “S8”, time line for alert source “S1” is the first showing critical time, as indicated by the portion 44 of that timeline. Furthermore, the issue indicated by the alerts in at time lines of “S1” and “S2” seems to be resolved at around the same time. The alert source presented at time line “S1” was critical for at least 3 time units at the 5 minute time unit of the zoomed timeline 38 of FIG. 6, i.e., at least 15 minutes.

FIG. 7 shows a still-further zoomed timeline 38, in which the selected time unit is now 1 minute (zoomed in from the 5 minute timeline of FIG. 6). As shown in FIG. 7, the issue indicated by the portion 46 of the time line for alert source “S5” was of a very short duration. The differently hatched part 46 (e.g., suitably color coded by light green, for example) has a duration of two minutes (as can be seen from the other brightly colored time units that are likely to each have a duration of one minute).

To create an efficient implementation of the timeline 38, a tree structure can be used, where each level relates to a different time unit. To make a clear distinction between a fraction of critical time of zero and a fraction of critical time that is near to zero, a darker shade or hue of green can be used for intervals for which the fraction is exactly zero and a brighter shade or hue of green can be used for intervals for which the fraction is low but above zero. In this way, one can quickly hover over large intervals of time for which there are no critical alerts. For example, to inspect the alerts 34 for a total period of one month, one can start at a zoomed-out level using as largest time units one week or one day. In this way, the days where there are not any critical intervals for all days of the complete month can quickly be determined, even if these critical intervals may have a duration of only a few seconds. Next, by zooming in on the days that do not have a dark green color, the displayed rectangle will be automatically refined to seeing time units of days or hours, where the hours that have a dark green color can be discarded, et cetera.

In addition to a horizontal hierarchy of time units, a vertical hierarchy of the alert sources can be defined, where, for example, the highest level is specified by the host and the second level by the alert type. Alternatively, a more detailed hierarchy can be defined using a hierarchy of alert types. The timelines 38 of multiple parameters can be grouped specified by an intermediate node of this vertical hierarchy. In this case, the timeline 38 for a given group of parameters will show the average critical fraction, averaged over the parameters of the group. With this vertical hierarchy, a rooted directed tree can be associated to the time line 38. To apply the vertical hierarchy, one can first select either the ‘fold’ or ‘unfold’ option, by selecting this option, for example, in a drop-down menu. Next one can (repeatedly) select one of the timelines 38 (by simply clicking on it). If ‘fold’ is selected, clicking on a timeline 38 will combine the given time line with all its siblings to obtain a combined time line. If ‘unfold’ is selected, clicking on a timeline 38 will unfold the given time line: provided that the timeline 38 does not correspond to a leaf node in the hierarchy tree, it will be replaced by a separate timeline 38 for each of its child nodes.

The disclosure has been described with reference to the preferred embodiments. Modifications and alterations may occur to others upon reading and understanding the preceding detailed description. It is intended that the exemplary embodiment be construed as including all such modifications and alterations insofar as they come within the scope of the appended claims or the equivalents thereof.

1. A non-transitory computer readable medium storing instructions readable and executable by an electronic processor to perform an alert monitoring method, the alert monitoring method comprising:
receiving alerts generated by one or more medical systems, each alert being timestamped and associated to the medical system that generated the alert;
segmenting a displayed time span into discrete time units whose size depends on the displayed time span;
generating a timeline representing the alerts over the displayed time span, the timeline comprising a two-dimensional grid of blocks with each block having a corresponding discrete time unit and a corresponding alert source for the one or more medical systems, and each block being color coded based on a fraction of the corresponding discrete time unit over which the corresponding alert is critical;
displaying, on a display device, the timeline representing the alerts over the displayed time span; and
in response to receiving a user input adjusting the displayed time span, repeating the segmenting, generating, and displaying to display an updated timeline representing the alerts over the updated displayed time span.

2. The non-transitory computer readable medium of claim 1, wherein the color coding of the blocks comprises:
color coding each block by inputting the fraction of the corresponding discrete time unit over which the corresponding critical alert is in a first range of a fraction-to-color mapping.

3. The non-transitory computer readable medium of claim 2, wherein the fraction-to-color mapping comprises:
color coding, with a first color, blocks whose fraction of the corresponding discrete time unit over which the corresponding critical alert is in a first range;
color coding, with a second color, blocks whose fraction of the corresponding discrete time unit over which the corresponding critical alert is in a second range.

4. The non-transitory computer readable medium of claim 3, wherein the fraction-to-color mapping further comprises:
color coding, with a third color, blocks whose fraction of the corresponding discrete time unit over which the corresponding critical alert is in a third range.

5. The non-transitory computer readable medium of claim 3, wherein the fraction-to-color mapping comprises further comprises:
color coding, with a zero-indicative color, blocks whose fraction of the corresponding discrete time unit over which the corresponding critical alert is equal to zero.

6. The non-transitory computer readable medium of claim 3, wherein the fraction-to-color mapping comprises further comprises:
color coding, with a max-indicative color, blocks whose fraction of the corresponding discrete time unit over which the corresponding critical alert is equal to unity.

7. The non-transitory computer readable medium of claim 2, wherein the fraction-to-color mapping is a continuous fraction-to-color mapping.

8. The non-transitory computer readable medium of claim 2, wherein the fraction-to-color mapping depends on the displayed time span.

9. The non-transitory computer readable medium of claim 1, wherein the alert monitoring method further comprises:
providing a zoom control via which a user can zoom the displayed timeline in or out, wherein the user input adjusting the displayed time span is received via the zoom control.

10. The non-transitory computer readable medium of claim 1, wherein the alert monitoring method further comprises:
receiving a user input modifying the alert sources by grouping two or more of the alert sources into a single alert source or breaking up an alert source into two or more constituent alert sources; and
repeating the segmenting, generating, and displaying to display an updated timeline representing the alerts with the modified alert sources.

11. The non-transitory computer readable medium of claim 1, wherein the alert monitoring method further comprises:
receiving a selection of a block via a user-operable input device; and
adjusting the color scale of the selected block.

12. The non-transitory computer readable medium of claim 11, wherein the adjusting comprises:
automatically changing the discrete time unit into which the parameter timelines are segmented based on a determined zoom level comprising the selected block.

13. The non-transitory computer readable medium of claim 1, wherein the alert monitoring method further comprises:
receiving a selection of a row of alerts of the displayed timeline via a user-operable input device;
grouping alerts corresponding to a component of one of the medical systems based on the selected row.

14. The non-transitory computer readable medium of claim 1, wherein the alert monitoring method further comprises:
receiving a selection of a block of the displayed timeline via a user-operable input device; and
displaying a list of the alerts received from the corresponding alert source.

15. The non-transitory computer readable medium of claim 1, wherein the alert monitoring method further comprises:
using the timeline to predict a failure or malfunction of one or more of the medical systems.

16. The non-transitory computer readable medium of claim 1, wherein the medical systems comprise Picture Archiving and Communication System (PACS) systems and the receiving of the alerts comprises receiving the alerts from the PACS systems.

17. An alert monitoring method, comprising:
receiving timestamped alerts generated by a plurality of alert sources;
segmenting a time span to be displayed into discrete time units whose size depends on the time span;
displaying, on a display, a timeline representing the alerts over the time span, the displayed timeline comprising blocks having corresponding discrete time units and alert sources, each block being displayed with a color determined based on a fraction of the corresponding discrete time interval over which the corresponding alert is critical;
receiving an adjusted time span to be displayed;
segmenting the adjusted time span into adjusted discrete time intervals whose size depends on the adjusted time span; and
displaying an updated timeline representing the alerts over the adjusted time span, each block of the updated timeline being displayed with a color determined based on a fraction of the corresponding adjusted discrete time interval over which the corresponding critical alert.

18. The method of claim 17, further comprising:
color coding each block by inputting the fraction of the corresponding discrete time interval over which the corresponding critical alert is in a first range of a fraction-to-color mapping.

19. A non-transitory computer readable medium storing instructions readable and executable by an electronic processor to perform an alert monitoring method, the alert monitoring method comprising:
segmenting a displayed time span into discrete time units whose size depends on the displayed time span;
generating a timeline representing alerts generated by alert sources over the displayed time span, each alert being timestamped and associated to the alert source that generated the alert, the timeline comprising a two-dimensional grid of blocks with each block having a corresponding discrete time unit and a corresponding alert source comprising one of the alert sources or a group of two or more of the alert sources, and each block being color coded based on a fraction of the corresponding discrete time unit over which the corresponding alert is critical;
displaying, on a display device, the timeline representing the alerts over the displayed time span; and
in response to receiving a user input adjusting the displayed time span, repeating the segmenting, generating, and displaying to display an updated timeline representing the alerts over the updated displayed time span.

20. The non-transitory computer readable medium of claim 19, wherein the method further comprises:
color coding each block by inputting the fraction of the corresponding discrete time interval over which the corresponding alert is critical is in a first range of a fraction-to-color mapping.
